(19) United States
(12) Patent Application Publication
(10) Pub. No.: US 2009/0110611 A1
(43) Pub. Date: Apr. 30, 2009

(54) Fan capable of giving fragrant smell

(76) Inventor: Wen-Hui Chen, Yongkang City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/979,081

(22) Filed: Oct. 31, 2007

(51) Int. Cl. A61L 9/12

(52) U.S. Cl. 422/124

(57) ABSTRACT

A fan includes a motor holding member, and a fragrance giving device; the motor holding member has a slot and several vent holes thereon; the fragrance giving device includes a first shell-shaped member, a second shell-shaped member, and a fragrant unit pressed between the first and the second shell-shaped members; the fragrance giving device is inserted in the motor holding member through the slot in order for the fragrant unit to face the vent holes; therefore, when the fan is running, it can give off fragrant smell, which will be carried with the wind produced by the fan, thus making the room full of fragrant air.

### BACKGROUND OF THE INVENTION

1. Field of the Invention

The present invention relates to a fan capable of giving fragrant smell, more particularly one, which includes a motor holding member, and a fragrance giving device inserted in the motor holding member; thus, when the fan is running, it can give off fragrance, which will be carried with the wind produced by the fan to make the room full of fragrant air.

2. Brief Description of the Prior Art

Manufacturers have to keep on developing new and novel products to attract consumers.

It is getting more difficult for single-functional products to sell well in the present market because consumers like to buy and use multifunctional products. Therefore, there are multifunctional electronic rice cookers, air conditioners, drinking fountains, business machines, electric fans etc in current markets.

### SUMMARY OF THE INVENTION

It is a main object of the present invention to provide a dual-functional fan, which will give off fragrance while operating to make the air cooler.

A fan according to an embodiment of the present invention includes a motor holding member, and a fragrance giving device, which is inserted in the motor holding member through a slot of the motor holding member. The fragrance giving device includes a first shell-shaped member, a second shell-shaped member, and a fragrant unit; the first and the second shell-shaped members have a middle hollowness; the fragrant unit is pressed between the shell-shaped members at a periphery so as to face the middle hollowness of the shell-shaped members. Therefore, when the fan is running, it can give off fragrance, which will be carried with the wind produced by the fan to make the room full of fragrant air.

### BRIEF DESCRIPTION OF THE DRAWINGS

The present invention will be better understood by referring to the accompanying drawings, wherein:

FIG. 1 is an exploded perspective view of the fragrance giving device of the present invention,

FIG. 2 is a side view of the fragrance giving device of the present invention (1),

FIG. 3 is a side view of the fragrance giving device of the present invention (2),

FIG. 4 is a front view of the fragrance giving device of the present invention, and

FIG. 5 is a partial side view of the fan of the present invention with the fragrance giving device being fitted to the motor holding member.

### DETAILED DESCRIPTION OF THE PREFERRED EMBODIMENTS

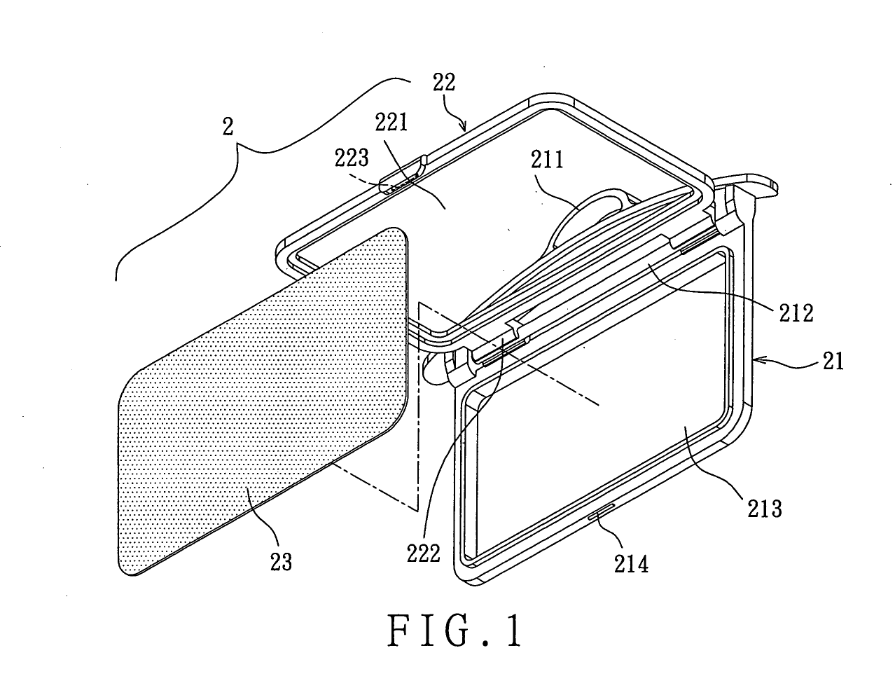

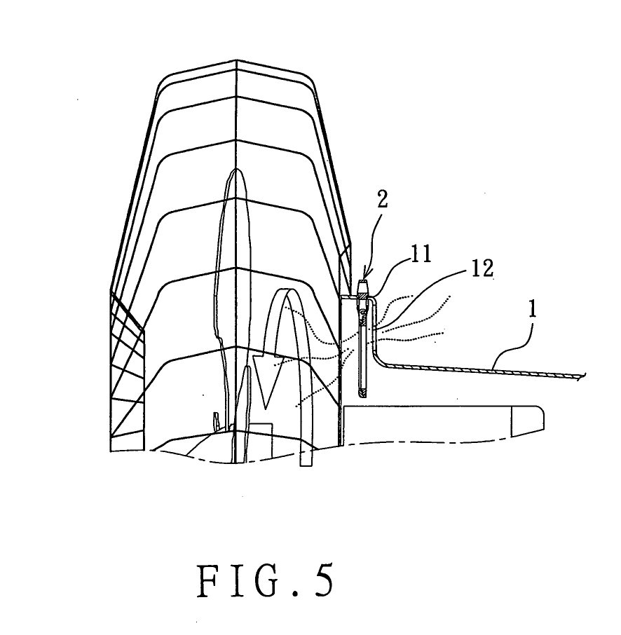

Referring to FIGS. 1 and 5, a preferred embodiment of a fan of the present invention includes a fragrance giving device 2, which is fitted to a motor holding member 1 of the fan.

The motor holding member 1 has a slot 11, and several vent holes 12 thereon. The fragrance giving device 2 is inserted in the motor holding member 1 through the slot 11 in a removable manner.

The fragrance giving device 2 comprises:

a first shell-shaped member 21, which has a handle 211 on a top, a connecting shaft 212 under the handle 211, a hollow portion 213, and a fastening piece 214 on a lower end;

a second shell-shaped member 22, which has a hollow portion 221, a connecting part 222 on a top, and a fastening piece 223 on a lower end thereof; the first and the second shell-shaped members 21 and 22 are pivoted together with the connecting shaft 212 of the first shell-shaped member 21 being received in the connecting part 222 of the second shell-shaped member 22; the first and the second shell-shaped members 21 and 22 will be fixed together after they are pivoted to be closely together with the fastening pieces 214 and 223 being joined to each other; the hollow portions 213 and 221 will face each other when the first and the second shell-shaped members 21 and 22 are fixed together; and

a fragrant unit 23, which is either a cotton sheet having already been soaked in essence or one having fragrance applied thereto; the fragrant unit 23 is tightly pressed between the first and the second shell-shaped member 21 and 22 at a periphery thereof so as to face the hollow portions 213 and 221 of the shell-shaped members 21 and 22.

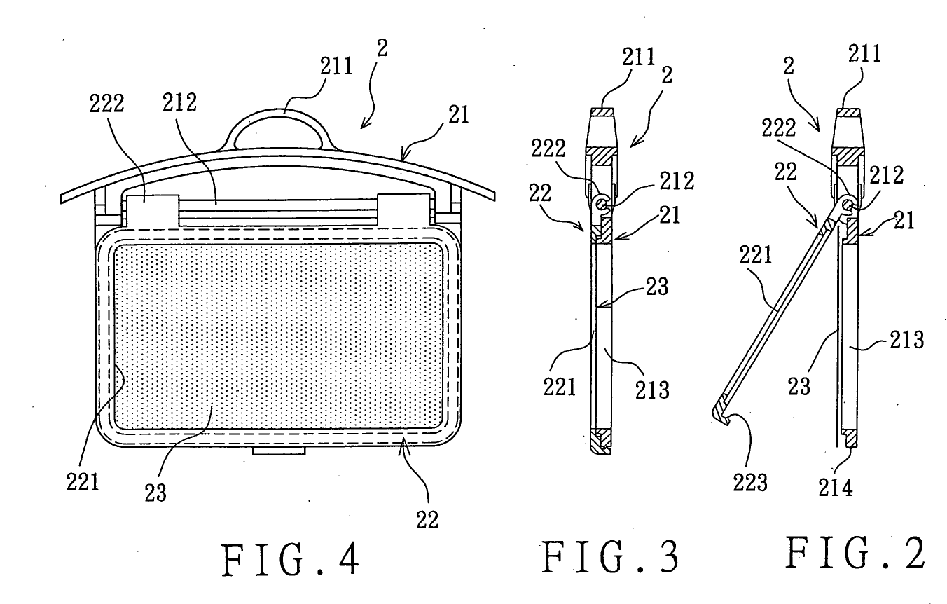

Therefore, when the fragrance giving device 2 is inserted in the motor holding member 1 of the fan through the slot 11, the fragrant unit 23 will face the vent holes 12. In use, referring to FIGS. 2, 3, and 4, first the fragrant unit 23 is positioned in one of the shell-shaped members 21 and 22, and the shell-shaped members 21 and 22 are fixed together with the fastening pieces 214 and 223 being joined to each other; thus, the fragrant unit 23 is pressed and fixed between the first and the second shell-shaped members 21 and 22 to form the fragrance giving device 2. Then, the fragrance giving device 2 is inserted in the motor holding member 1 through the slot 11 in order for the fragrant unit 23 to face the vent holes 12 of the motor holding member 1; thus, when the fan is running, it will give off fragrant smell, which will be carried to the users with the wind produced by the fan. Consequently, the room will be full of fragrant air.

Furthermore, the user is allowed to hold the handle 211 to easily lift the fragrance giving device 2 out of the motor holding member 2, and the first and the second shell-shaped members 21 and 22 can be easily pivoted to an open position. Therefore, the fragrant unit 23 can be easily replaced with a new one, and essence/fragrance can be applied to the fragrant unit 23 after the essence/fragrance on the fragrant unit 23 has run out.

From the above description, it can be seen that the present invention has the following advantages:

1. Because the fan of the present invention includes the fragrance giving device, it can give off fragrant smell in operation, which will be carried with the wind produced by the fan, thus making the room full of fragrant air.

2. The fan is convenient to use because the fragrance giving device can be easily lifted out of the motor holding member, and can be easily opened for changing the fragrant unit/applying essence to the fragrant unit.

What is claimed is:

1. A fan capable of giving fragrant smell, comprising
a motor holding member, the motor holding member having a slot thereon; and
a fragrance giving device, which is inserted in the motor holding member through the slot; the fragrance giving device including a first shell-shaped member, a second shell-shaped member, and a fragrant unit securely pressed between the first and the second shell-shaped members.

2. The fan capable of giving fragrant smell as claimed in claim 1, wherein the motor holding member has a plurality of vent holes to face the fragrant unit of the fragrance giving device.

3. The fan capable of giving fragrant smell as claimed in claim 1, wherein the first shell-shaped member has a handle.

4. The fan capable of giving fragrant smell as claimed in claim 3, wherein the first shell-shaped member has a connecting shaft under the handle, and the second shell-shaped member has a connecting part on a top; the first and the second shell-shaped members being pivoted together with the connecting shaft of the first shell-shaped member being received in the connecting part of the second shell-shaped member.

5. The fan capable of giving fragrant smell as claimed in claim 4, wherein each of the first and the second shell-shaped members has a fastening piece on a lower end thereof, and the first and the second shell-shaped members are fixed together by means of the fastening pieces.

6. The fan capable of giving fragrant smell as claimed in claim 1, wherein the fragrant unit is a cotton sheet having fragrance applied thereto.

7. The fan capable of giving fragrant smell as claimed in claim 1, wherein each of the first and the second shell-shaped members has a fastening piece on a lower end thereof, and the first and the second shell-shaped members are fixed together by means of the fastening pieces.
